(19) United States
(12) Patent Application Publication
(10) Pub. No.: US 2015/0005183 A1
(43) Pub. Date: Jan. 1, 2015

(54) PROTEIN BIOMARKERS OF LATE STAGE BREAST CANCER

(71) Applicants: David Krizman, Gaithersburg, MD (US); Marlene M. Darfler, Rockville, MD (US); Thomas P. Conrads, Rockville, MD (US); Brian L. Hood, Rockville, MD (US)

(72) Inventors: David Krizman, Gaithersburg, MD (US); Marlene M. Darfler, Rockville, MD (US); Thomas P. Conrads, Rockville, MD (US); Brian L. Hood, Rockville, MD (US)

(73) Assignee: EXPRESSION PATHOLOGY, INC., Rockville, MD (US)

(21) Appl. No.: 13/932,990

(22) Filed: Jul. 1, 2013

(51) Int. Cl. G01N 33/68

(52) U.S. Cl. CPC G01N 33/6893
USPC 506/9; 435/23; 436/501; 435/7.92; 530/350; 530/386; 435/6.12; 435/6.11; 435/190; 530/356; 435/219

(57) ABSTRACT

This patent application discloses and describes proteins found to be differentially expressed between primary tumor breast cancer cells histologicaly defined as early stage (stage 0) breast cancer and primary breast cancer cells histologicaly defined as late stage (stage 3) breast cancer. These proteins can be used either individually or in specific combinations in diagnostic and prognostic protein assays on various biological samples from breast cancer patients to indicate the that a breast cancer patient's cancer is in an early, non-aggressive stage or in a late, aggressive stage. Determination of differential expression of these proteins can also be useful for indicating additional therapies to combat the aggressiveness of late stage breast cancer. The full length intact proteins can be assayed or peptides derived from these proteins can be assayed as reporters for these proteins. These proteins can also be identified as “companion diagnostic” proteins, wherein the differentially expressed proteins that are used as diagnostic and prognostic indicators can also be used as targets for therapeutic intervention of breast cancer. Also disclosed and described herein are isotope labeled versions of peptides from the proteins.

This application claims the benefit of U.S. Provisional Application No. 61/428,160, filed Dec. 29, 2010, entitled “Protein Biomarkers of Late Stage Breast Cancer,” the contents of which are hereby incorporated by reference in their entirety.

### BACKGROUND

In the United States, an estimated 180,000 new cases of invasive breast cancer are diagnosed among women on an annual basis, and approximately 40,000 are expected to die from breast cancer yearly. Only lung cancer accounts for more cancer deaths in women. Based on the most recent data, relative survival rates for women diagnosed with breast cancer are 89% survival 5 years after diagnosis, 81% after 10 years, and 73% after 15 years. However five-year relative survival is lower among women with a more advanced stage (more aggressive) at diagnosis where the 5-year relative survival is 98% for localized disease (stage 0 and 1), 84% for regional disease (stage 2), and 27% for distant-stage disease (stage 3 and 4). Thus, providing the ability to identify those patients at greater risk of having later stage cancer (stage 3 and 4) that may at first appear to be early stage cancer (stage 0-2) is paramount to increasing survival rates. This is because enhanced, more informed treatment decisions can be made based on identifying, at an earlier point, those patients that harbor more aggressive disease, which will ultimately save lives.

Once breast cancer is diagnosed in a patient, a typical initial treatment is to remove the tumor by surgery followed secondarily by chemotherapy treatment designed to kill any residual cancer cells not removed by surgery. Knowledge of the stage of the breast cancer is critical to patient treatment because different stages/grades of breast cancer respond differently to different treatment strategies. Determining the stage, grade, and/or aggressiveness of breast cancer is best determined by analyzing the actual breast tumor tissue after removal from the patient. Tumor cells within the breast tumor tissue can be histologicaly and molecularly analyzed in order to determine grade, stage, and/or extent of breast cancer as well as identify which therapeutic agent is best to use against any tumor cells that remain in the patient. The most widely and advantageously available form of cancer patient tissue is formalin fixed, paraffin embedded tissue.

Formaldehyde/formalin fixation of surgically removed tissue is by far and away the most common method of preserving cancer tissue worldwide and is the accepted convention for standard pathology practice. Aqueous solutions of formaldehyde are referred to as formalin. Formaldehyde/formalin fixation typically employs aqueous solutions of formaldehyde referred to as formalin. “100%” formalin consists of a saturated solution of formaldehyde (about 40% formaldehyde by volume or 37% by mass) in water, with a small amount of stabilizer, usually methanol to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Thus molecular analytical methods to analyze formalin fixed cancer tissue will be the most accepted and heavily utilized methods for analysis of cancer patient tissue.

A critical issue for determining breast cancer treatment is to identify those patients who at first appear to harbor non-aggressive localized disease (stage 0-2) that may actually harbor more aggressive disease (stage 3-4) that will more than likely recur despite surgery and first-line chemotherapy treatment. If patients can be better identified whose disease will more than likely recur because it is actually a more aggressive form of breast cancer than my appear from histopathology or other measures, then more aggressive surgical (e.g., radical mastectomy as opposed to tylectomy aka “lumpectomy”), first line chemotherapy, or an additional second line of therapy can be performed on those patients.

There are existing molecular tests designed to identify patients whose breast cancers are more aggressive than others by analyzing patient-derived formalin fixed tissue, such as the OncotypeDx test from GenomicHealth and the Mammaprint test from Agendia. However, these tests result in large numbers of patients that fall into an intermediate category where the test cannot give an indication of the likelihood of disease recurrence or non-recurrence. In addition, existing tests analyze nucleic acids and not the actual functional entities, proteins, that are differentially present in the breast cancer tissue/cells. Tests that utilize proteins as indicators of aggressive forms of breast cancer are more advantageous because it is the proteins, not the nucleic acids, that principally do the work of the cell, and it is the aberrantly expressed proteins that cause a cell to become cancerous. In addition, aberrantly expressed proteins can be targeted by drugs to selectively or specifically attack the cancer cells. Thus, diagnostic tests that analyze proteins, and proteomic technologies to perform analysis, are advantageous.

The field of proteomics strives to establish the identities, quantities, structures, and biochemical and cellular functions of all proteins in an organism. Application of proteomics has historically proceeded mostly on a one-protein-at-a-time basis. The human proteome contains hundreds of thousands of proteins, and using recently developed proteomic techniques, changes in proteins that are over expressed in cells within solid tissue as well as proteins that are shed into body fluids throughout disease progression can now be examined. Specific proteins, and patterns of proteins, that are found to be differentially expressed in diseased cells vs. normal cells can be reflective and diagnostic of a given disease state.

In recent years, advanced technologies and methodologies have been developed that provide an interface between clinical medicine/pathology and proteomics. High throughput global proteomic analysis technologies such as liquid-chromatography-tandem mass spectroscopy (LC-MS/MS) can be used to generate proteomic profiles from biological samples which are specific for disease. Such global profiles can be performed on all types of biological samples including frozen tissue, formalin fixed tissue, and bodily fluids.

Without targeted, convenient, and reliable screening/diagnostic tests for cancer, the lack of molecular diagnostic assays will continue to plague the health care system and complicate efforts to detect and treat malignancies in their earliest stages. Breast cancer protein biomarkers that are differentially expressed in early stage vs. late stage aggressive breast cancer tissue would help in reducing the suffering of women from breast cancer by greatly improving diagnosis of breast cancer, provide for improved diagnostics and prognostic capabilities, and provide targets for development of drugs that can more effectively treat breast cancer. In addition, the presence of these biomarkers in bodily fluids that result from localized shedding into the breast tissue lumens, and ultimately into blood would present a readily accessible body fluid that can be sampled for proteomics-based screening and early detection. The development of a proteomics-based diagnostic/screening test and treatment strategies for early vs. late stage breast cancer would represent a significant medical advance for a “personalized medicine” approach to breast cancer diagnosis, prognosis, and therapy.

### SUMMARY

The present disclosure provides, among other things, a method of diagnosing the presence of late stage (stage 3) breast cancer disease that may be masked by its histological appearance as a less-aggressive stage (stage 0) form of the disease. A sample is obtained from a patient. The sample is breast cancer tissue, breast cancer cells, or a bodily fluid such as serum or fluid aspirate that may contain cells/proteins derived from a patient's cancerous tissue. The presence and level of expression of at least one, two, three, four, five, six, seven, eight or more of the proteins listed in Table 1 are determined in the sample. The level of expression of the detected proteins in late stage (stage 3) is compared to the level of expression of the same proteins in early stage (stage 0) breast cancer. The differential expression of at least one or more proteins, or combinations of multiple proteins indicates the presence of late stage (stage 3) breast cancer disease. In this way a prognosis can be made, which is to determine that a breast cancer is late stage (stage 3), which may indicate a different treatment strategy for late stage (stage 3) vs. early stage (stage 0). In one embodiment, proteins, or peptide fragments thereof, are detected by mass spectroscopy, and the level of expression of at least one or more than one of the proteins is determined by a spectral count quantization mass spectrometry or by Selected Reaction Monitoring (SRM) mass spectrometry; which can also be referred to as a Multiple Reaction Monitoring (MRM) mass spectrometry, collectively referred to hereinafter referred to as SRM/MRM assay(s). In another embodiment, the proteins are detected and their levels of expression are determined by a protein microarray or by an immunoassay.

This disclosure also provides a method of identifying protein targets for therapeutic intervention in breast cancer. The presence and level of expression of one, two, three, four, five, six, seven, eight or more of the proteins listed in Table 1 are detected in the sample. The level of expression of the detected proteins in early late stage (stage 3) breast tissue is compared to the level of expression of the same proteins in early stage (stage 0) breast cancer. The differential expression of one, two, three, four, five, six, seven, eight or more proteins may indicate choice of therapy and define specific targets for therapeutic intervention in breast cancer.

The choice of sample for assessing protein expression includes solid tissue (normal or diseased) and bodily fluids derived from the patient through surgical means including biopsy and aspiration. Protein expression is most advantageously detected and measured in cells or tissue samples from solid tumor tissue because these are the actual cells that are growing and causing the disease. However, it is sometimes less invasive and more comfortable for the patient to collect a bodily fluid such as blood and/or ascites fluid that surrounds the tumor itself. These fluid sources may contain a number of the proteins listed in Table 1 because they can be secreted by the tumor cells into the surrounding fluid or the tumor cells themselves become dislodged from the solid tumor and can now be found in the fluid, and which in many cases is an easier sample to collect from a breast cancer patient. The proteins listed in Table 1 can be detected and levels measured in either solid tissue or a bodily fluid from the breast cancer patient.

In one embodiment a collection of biomarkers is provided for diagnosing whether or not a breast cancer is early stage (stage 0) or late stage (stage 3) comprising the steps of:
(a) measuring the level of expression of one, two, three, four, five, six, seven, eight or more of the proteins listed in Table 1 in a sample from a human patient, in which said sample comprises breast cancer tissue, breast cancer cells, or a bodily fluid such as blood or ascites fluid containing proteins from said patient's breast cancer said sample; and
(b) determining increased expression and/or decreased expression of said one, two, three, four, five, six, seven, eight or more of the proteins listed in Table 1 in a late stage (stage 3) breast cancer as compared to expression levels of those proteins listed in Table 1 in an early stage (stage 0) breast cancer;
   wherein the identification of one, two, three, four, five, six, seven, eight or more of those proteins indicates the potential that a primary breast cancer is more likely a late stage (stage 3) vs. an early stage (stage 0) in said patient. In another embodiment of the diagnostic method, one, two, three, four, five, six, seven, eight or more of the proteins listed in Table 1 as undergoing an increase are examined. In yet another embodiment of the diagnostic method, one, two, three, four, five, six, seven, eight or more of the proteins listed in Table 1 as undergoing a decrease are examined. In still another embodiment of the diagnostic method, one, two, three, four, five, six, seven, eight or more of the proteins listed in Table 1 as undergoing an increase, in combination with one, two, three, four, five, six, seven, eight or more of the proteins listed in Table 1 as undergoing a decrease are examined. The greater the number of biomarker proteins found in Table 1 to be increased (over-expressed) and/or decreased (under-expressed) in a sample obtained from a patient, the higher the probability that a primary breast cancer is a late stage (stage 3) cancer in that patient.

Certain embodiments of the invention are described below.
1. A method of diagnosing that a breast cancer as an early stage primary breast cancer (stage 0) or a late stage (stage 3) breast cancer comprising the steps of:
   a) measuring the level of expression of at least one or more, at least two or more, at least 3 or more, or multiples and combinations of the proteins listed in Table 1 in a sample from a human patient, in which said sample comprises breast cancer tissue, breast cancer cells, or a bodily fluid such as blood or ascites fluid containing proteins from said patient's breast cancer said sample; and
   b) determining increased expression and/or decreased expression of said at least one or more, at least two or more, at least 3 or more, or multiples and combinations of the proteins listed in Table 1 in a late stage (stage 3) breast cancer as compared to expression levels of said at least one or more, at least two or more, at least 3 or more, or multiples and combinations of the proteins listed in Table 1 in early stage (stage 0) breast cancer indicating the potential that a primary breast cancer is more or less aggressive in said patient.
2. The method of embodiment 1, wherein said breast cancer sample consists essentially of breast epithelial cells.
3. The method of embodiment 1, wherein said bodily fluids include but are not limited to fractionated or unfractionated blood, serum, plasma, lymphatic fluid, or fluid collected by pleural effusion.
4. The method of embodiment 1, wherein the tissue is collected by biopsy or surgical procedure.
5. The method of embodiment 4, wherein the tissue is chemically fixed and preserved.
6. The method of embodiment 5, wherein said chemical fixation and preservation comprises formalin fixation and embedding in paraffin.
7. The method of embodiments 4 or 5, wherein the tissue is frozen.
8. The method of embodiment 1, wherein said proteins are measured as intact, full-length proteins or are measured by measuring multiple or individual peptides derived by fragmentation of the intact, full-length proteins.
9. The method of any of embodiments 1-8, wherein said proteins are detected by mass spectroscopy and the level of measured expression of said proteins is determined by spectral count quantification after said mass spectroscopy
10. The method of any of embodiments 1-8, wherein said proteins are detected by mass spectroscopy and the level of measured expression of said proteins is determined by a Selected Reaction Monitoring (SRM) assay.
11. The method of any of embodiments 1-8, wherein said proteins are detected by mass spectroscopy and the level of measured expression of said proteins is determined by a multiplex SRM assay, termed a multiple reaction monitoring (MRM) assay where more than one protein is detected and quantitated in a single mass spectrometry analysis.
12. The method of any of embodiments 8-11, wherein said mass spectroscopy is selected from the group consisting of LC-ESI-MS/MS, MALDI-MS, tandem MS, TOF/TOF, TOF-MS, TOF-MS/MS, triple quadrupole MS, and triple quadrupole MS/MS.
13. The method of embodiment 12, wherein said mass spectroscopy comprises liquid chromatography-tandem mass spectroscopy.
14. The method of any one of any of embodiments 1-88, wherein said proteins are detected and their levels of expression are determined by a protein microarray or by an immunoassay.
15. The method of embodiment 14, wherein said immunoassay is selected from the group consisting of immunohistochemistry, Western blot, dot blot, and ELISA.
16. A method of indicating choice of therapy of primary breast cancer, comprising the steps of:
   a) detecting the presence and measuring the level of expression of at least one or more, at least two or more, at least 3 or more, or multiples and combinations of the proteins listed in Table 1 in a sample from a human patient, in which said sample comprises breast cancer tissue, breast cancer cells, or a bodily fluid such as blood or ascites fluid containing proteins from said patient's breast cancer said sample; and
   b) determining increased expression and/or decreased expression of said at least one or more, at least two or more, at least 3 or more, or multiples and combinations of the proteins listed in Table 1 in a late stage (stage 3) breast cancer as compared to expression levels of said at least one or more, at least two or more, at least 3 or more, or multiples and combinations of the proteins listed in Table 1 in early stage (stage 0) breast cancer indicating the potential that a primary breast cancer is more or less aggressive in said patient.
17. A method comprising quantifying the amount of one or more, two or more, three or more, four or more, five or more, six or more, seven or more, or eight or more of the proteins in Table 1 or peptide fragments thereof.
18. A composition comprising one or more, two or more, three or more, four or more, five or more, six or more, seven or more, eight or more, or ten or more of the proteins in Table 1, peptides thereof, and/or antibodies thereto.
19. The composition of embodiment 18, comprising one or more, two or more, three or more, four or more, five or more, six or more, seven or more, or eight or more peptides of proteins in Table 1, wherein each peptide is derived from a different protein.
20. The composition of embodiment 19, wherein each of the peptides is labeled with one or more isotopes independently selected from the group consisting of: 18O, 17O, 34S, 15N, 13C, 2H or combinations thereof.
21. The composition of any of embodiments 19-20, comprising one or more, two or more, three or more, four or more, five or more, six or more, seven or more, or eight or more peptides of proteins in Table 1 that are increased in tissues from primary tumors that are late stage (stage 3) breast cancers.
22. The composition of any of embodiments 19-21, comprising one or more, two or more, three or more, four or more, five or more, six or more, seven or more, or eight or more peptides of proteins in Table 1 that are decreased in tissues from primary tumors that are late stage (stage 3) breast cancers.
23. The composition of any of embodiments 18-22, wherein said composition is substantially pure or free of other cellular components selected from any combination of other proteins, membranes lipids and/or nucleic acids.
24. The method of any of embodiments 1-17, further comprising assessing and/or determining the level (amount) or sequence of one, two, three, four, five, six, seven, eight nine, ten or more nucleic acids in said protein digest.
25. The method of embodiment 24, wherein said nucleic acids have a length selected independently from greater than about 15, 20, 25, 30, 35, 40, 50, 60, 75, or 100 nucleotides in length.
26. The method of embodiment 25, wherein said nucleic acids have a length selected independently from less than about 150, 200, 250, 300, 350, 400, 500, 600, 750, 1,000, 2,000, 4,000, 5,000, 7,500, 10,000, 15,000, or 20,000 nucleotides in length.
27. The method of any of embodiments 24-26, wherein assessing and/or determining the level (amount) or sequence comprises, determining either the sequence of nucleotides in the nucleic acids and/or a characteristic of the nucleic acids by any one or more of: nucleic acid sequencing, conducting restriction fragment polymorphism analysis, conducting hybridization with another nucleic acid, identification of one or more deletions and/or insertions, and/or determining the presence of mutations, including but not limited to, single base pair polymorphisms, transitions and/or transversions.
28. The method of any of any of embodiments 24-27, wherein one, two, three, four, five, six, seven, eight nine, ten or more nucleic acids encode for proteins in Table 1.
29. The method of embodiments any of 24-28, wherein said nucleic acids encode for proteins of SEQ ID Nos: 1-50, 51-113, 1-25, 26-50, 51-75, 76-100, 1-10, 11-20, 21-30, 31-40, 41-50, 51-60, 61-70, 71-80, 81-90, 91-100, 101-113 or fragments thereofable 1.

### DETAILED DESCRIPTION

### Biomarkers

Methodologies at the interface between clinical medicine/pathology and proteomics were utilized to identify differentially expressed proteins between early stage (stage 0) breast cancer epithelial cells and late stage (stage 3) breast cancer epithelial cells. The list of proteins of proteins provided in Table 1 was determined by global LC-MS/MS proteomic profiling of cells obtained from early stage (stage 0) breast cancer tissue and late stage (stage 3) breast cancer tissue; and comparing those proteins that were consistently over-expressed or under-expressed in early stage (stage 0) breast cancer cells as compared to late stage (stage 3) breast cancer cells. Of note is that many or all of these proteins may be readily assayed in bodily fluids that derive from breast cancer cells, such as ascites fluid or fluids derived from blood such as plasma and serum. It is either breast-derived tissue, breast epithelial cells, or bodily fluids that would be assayed for diagnostic evaluation of breast cancer by assaying for specific protein expression from the list described herein. Also, one or more of the same proteins form the basis for a targeted therapeutic approach whereby a drug would be directed towards these proteins. Identification of these proteins provides for the ability to determine early stage (stage 0) breast cancer from late stage (stage 3) in a broad variety of biological samples collected from a subject, including fixed and frozen tissue, and bodily fluid samples derived from both blood and ascites fluids. The diagnostic and prognostic endpoint for disease analysis includes both single analytes and proteomic patterns. Proteomic patterns may be composed of many individual proteins, each of which may not individually identify early stage (stage 0) from late stage (stage 3) breast cancers, but collectively identify early stage (stage 0) from late stage (stage 3) breast cancers. Also provided are individual proteins, patterns of proteins, and/or collections of multiple proteins to be utilized for diagnosis, prognosis, and therapy of recurrent breast cancer.

The methods provided herein make possible the evaluation of a primary breast cancer's stage of progression (early vs. late) and treatment strategies for a subject (patient) with breast cancer. The methods are useful for determining if a breast cancer that appears to be early stage non-aggressive (stage 0) by visual histological methods is likely to be a more aggressive advanced stage (stage 3) of breast cancer. By measuring one, two, three, four, five, six, seven, eight or more of the proteins from the list of proteins in Table 1, breast cancer can be diagnosed in a subject, the prognosis of that subject can be determined, and the specific drug for that subject's disease can be chosen. A sample of tissue, such as that which is surgically procured or biopsied from a subject and frozen or chemically fixed, or a bodily fluid, such as blood, serum, plasma, and/or ascites fluid is examined to evaluate and measure protein expression.

Observed differences in proteins from the list of proteins in Table 1 found in a biological sample from a subject with breast cancer that is early stage (stage 0) vs. a biological sample from a subject where the breast cancer is late stage (stage 3) represents a disease protein profile and is indicative of the presence, absence, nature or extent of cancer pathology in the patient.

In one embodiment, the difference between the late stage (stage 3) breast cancer protein profile and the reference early stage (stage 0) breast cancer protein profile comprises a difference in the amount of one, two, three, four, five, six, seven, eight or more biomarker proteins from the list in Table 1. The method for evaluating breast cancer pathology in a subject includes discriminating between different disease states or between a disease state and normal state. Such a profile is also used to determine prognosis, which aims to monitor the extent and expectations of the progression or regression of breast cancer in a given subject. To this end, the late stage (stage 3) breast cancer protein profile can be derived from a biological sample previously obtained from the subject, for example a biological sample obtained prior to treatment or as part of a general health screening.

The method is also well-suited to evaluate the efficacy of treatment decisions, such as drugs or surgeries. In the case of choice of drug therapy, one or more of the proteins within the late stage (stage 3) breast cancer protein profile can serve as a target for drug treatment. In one embodiment, the drug specifically interacts with individual and specific proteins from the list of proteins in Table 1. In another embodiment, the drug interacts with a binding partner of a protein from the list in Table 1, thereby altering the ability of the protein in Table 1 to interact with its binding partner or to carry out its biological function. In still another embodiment, the expression profile of one, two, three, four, five, six, seven, eight or more of the proteins may be used to select the drug therapy, and/or the duration/regimen.

The method further comprises a classification model or algorithm, based on one or more protein differences from the protein list of Table 1 between the test protein profile of a biological sample from a subject suspected of late stage (stage 3) breast cancer and the reference protein profile from a biological sample from a subject having early stage (stage 0) breast cancer.

In some embodiments early stage (stage 0) or late stage (stage 3) breast cancer protein profiles or both are generated using mass spectrometry. In such embodiments the methods of mass spectrometry employed may advantageously use ion trap instruments or triple quadrupole instruments. Generally for analysis by mass spectrometry, full length intact proteins are reduced to individual peptides by treatment of protein samples with a proteolytic enzyme, e.g. trypsin, papain, chymotrypsin, and others, thus rendering a complex protein sample preparation to a complex lysate consisting of peptides. Such peptide lysates are the preferred form of sample for analysis of proteins from a biological sample by mass spectrometry, where the quantitative presence of specific and individual peptides is indicative of the quantitative presence of the full length intact proteins from which the peptides derive. In one embodiment, analysis of all peptides simultaneously in a global fashion may advantageously be performed on an ion trap mass spectrometry instrument. In one embodiment, analysis of targeted peptides that specifically focus assays on individual and specific peptides, and thus the proteins from which they derive, is conducted on a triple quadrapole mass spectrometry instrument. Performing targeted quantitative protein analysis by triple quadrupole mass spectrometry may be accomplished using SRM/MRM methodology. That methodology can be used to generate a protein profile to investigate the likelihood of late stage (stage 3) breast cancer in a subject from which a biological sample was obtained.

Prior to analysis by mass spectrometry, peptides in the lysates may be subject to a variety of techniques that facilitate their analysis and measurement by mass spectrometry. In one embodiment, the peptides may be separated by an affinity technique, such as immunologically-based purification (e.g., immunoaffinity chromatography), chromatography on ion selective media, or if the peptides are modified, by separation using appropriate media, such as lectins for separation of carbohydrate modified peptides. In one embodiment, the SISCAPA method, which employs immunological separation of peptides prior to mass spectrometric analysis is employed. The SISCAPA technique is described, for example, in U.S. Pat. No. 7,632,686. In other embodiments, lectin affinity methods (e.g., affinity purification and/or chromatography may be used to separate peptides from a lysate prior to analysis by mass spectrometry. Methods for separation of groups of peptides, including lectin-based methods, are described, for example, in Geng et al., J. Chromatography B, 752:293-306 (2001). Immunoaffinity chromatography techniques, lectin affinity techniques and other forms of affinity separation and/or chromatography (e.g., reverse phase, size based separation, ion exchange) may be used in any suitable combination to facilitate the analysis of peptides by mass spectrometry.

Another assay method includes immobilizing the proteins and/or peptides from the proteins, on a microarray (e.g., using immobilized antibodies) prior to detecting the proteins using antibody-based methods including sandwich-type assays. Other assay methods include immunohistochemical analysis utilizing antibody-based protein detection methods on thin tissue sections, where the proteins are maintained in full length (not subject to proteolysis) within the tissue section. Still other assay methods include antibody-based Western blot and ELISA protein detection methods, where the protein preparations interrogated are full length intact proteins and/or derivative peptides. All of these described protein detection methods may be used to detect individual polypeptides that derive from whole intact proteins, and thus these methods do not necessarily require the detection of whole intact proteins, but can involve the detection of peptides derived from the whole intact proteins. These methods may be used alone or in any combination, including in combination with mass spectroscopy based methods. Any suitable report/detection system known in the art may be employed with such assays including, but not limited to, fluorescence, UV/Vis chromatophore development, plasmon resonance, metal staining, and the like.

Accordingly, a useful method is provided for detecting proteins from the protein list in Table 1 and polypeptides derived from these proteins. The presence, absence, nature or extent of breast cancer pathology indicating late stage (stage 3) breast cancer disease in a patient can be evaluated in view of the expression of one or more expressed biomarker proteins from the list, and/or a derivative peptide or peptides from the same proteins.

In yet another aspect, a method is provided for screening a patient or population of patients for breast cancer by assaying for the presence of one or more proteins found in Table 1, or their derivative peptides. The assay(s) employed may include mass spectrometric assays, immunologic assays, such as a Western blot, enzyme linked immunosorbent assay (ELISA), or immunohistochemical methods on intact tissue sections, or any combination thereof. As noted above, plurality (e.g., one, two, three, four, five, six, seven, eight or more) of proteins or derivative peptides that increase or decrease with an increased likelihood of breast cancer recurrence can be analyzed, thereby increasing the predictive power of the screening assay. In one embodiment one, two, three, four, five, six, seven, eight or more of the proteins listed in Table 1 as undergoing an increase, in combination with one, two, three, four, five, six, seven, eight or more of the proteins listed in Table 1 as undergoing a decrease are examined.

### Identifying the Biomarkers

The protein biomarkers (e.g., the proteins in Table 1) were selected based on their differential patterns of expression observed in breast cancer epithelial cells obtained from histologicaly determined early stage (stage 0) primary tumors and breast cancer epithelial cells obtained from primary tumors that were histologicaly determined early stage (stage 0) breast cancer. Levels of some proteins were increased in cancerous cells obtained from early stage (stage 0) breast cancer tissue when compared to levels of the same proteins in late stage (stage 3) cancerous cells while levels of other proteins decreased in early stage (stage 0) cancer tissue cells as compared to levels in late stage (stage 3) cancerous cells.

Data present in Table 1 were collected by the mass spectrometry analysis of protein lysates from tissues and cells of patients that suffered from late stage (stage 3) breast cancer and early stage (stage 0) breast cancer. Protein lysates obtained from the cells of the two patient populations contain all the necessary information about differential protein expression. Protein lysates from the cells of those patient populations were prepared using the Liquid Tissue™ protocol and reagents. The preparation method included collecting cells (tissue sample) into a tube via tissue microdissection followed by maintaining the cells (tissue sample) at an elevated temperature in a buffer for an extended period of time. (e.g., from about 80° C. to about 100° C. for a period of time from about 10 minutes to about 4 hours) to reverse or release protein cross-linking. The buffer employed is a neutral buffer, (e.g., a Tris-based buffer, or a buffer containing a detergent) and advantageously is a buffer that does not interfere with mass spectrometric analysis. Once the formalin induced cross linking has been negatively affected, the cells are then digested to completion in a predictable manner using a protease (e.g., trypsin). The result of the heating and proteolysis is a liquid, soluble, dilutable biomolecule lysate.

The prepared the lysates were then analyzed by global proteomic mass spectrometry and the data is initially presented as identification of the total number of peptides in each protein lysate. Once as many peptides as possible were identified in a single MS analysis of a single lysate, then that list of peptides was compared to the list of peptides identified across all lysates in a study set. Thus, the starting point for determining differential protein expression by mass spectrometry was a list of peptides found to be expressed in one sample and/or group of similar samples and compared to the list of peptides found expressed in a second sample and/or group of similar samples. The first group of seven (7) Liquid Tissue™ samples were derived from early stage (stage 0) primary breast cancer tissue from while the second group of nine (9) Liquid Tissue™ samples were derived from late stage (stage 3) primary breast cancer tissue. The comparison of those proteins that were differentially expressed between these two groups of patients, early stage (stage 0) breast cancer vs. late stage (stage 3) breast cancer, formed the initial study set of proteins set forth in Table 1.

The classification of differential protein expression from the lists of peptides found in patients that suffered recurrent and those that did not suffer from recurrent breast cancer was accomplished by first determining which proteins were represented by a given list of peptides, and then to count the total number of peptides identified for each protein. That method of data collating is known as the Spectral Count method (SC). The spectral count for a given protein is thus based on the total number of peptides identified for that protein in a single lysate, which is a relative indicator for the abundance of that protein in the lysate that was analyzed by MS. Spectral count is a mathematical method that provides the ability to compare relative protein abundances for a given protein from one sample and/or group of similar samples to the next sample and/or group of similar samples. This approach can also be uses to distinguish protein abundance between individual proteins within a given sample.

Spectral counts between thousands of individual proteins are compared for samples obtained from breast cancer epithelial cells obtained from multiple primary patient-derived tumors that gave rise to recurrent breast cancer and breast cancer epithelial cells obtained from multiple primary patient-derived tumors that did not give rise to recurrent breast cancer.

The protein abundance was thus derived by mass spectrometry analysis of protein lysates from multiple breast cancer tissues using spectral counting (SC) of peptides. In addition, peptides whose sequences mapped to multiple protein isoforms were grouped as per the principle of parsimony. To determine statistically significant changes in protein abundance across patient samples by disease stage sub-groups, a hierarchical supervised cluster analysis of peptides identified from early stage (stage 0) versus late stage (stage 3) patient samples was performed in which the variance in total spectral count peptides identified was determined utilizing the Mann-Whitney rank-sum test (significance level p≦0.05, Fisher's exact test) paired with the filter criteria requiring that 60% of the samples in a supervised group had a minimum peptide count of two (2) or greater for a given protein.

Selection of the proteins in Table 1 was limited to those proteins that showed significantly (significance level p≦0.05, Fisher's exact test) higher or lower spectral count abundance in early stage (stage 0) breast cancer tissues vs. late stage (stage 3) breast cancer tissues.

Table 1 shows the names of 113 proteins, 32 that were increased and 81 decreased in abundance, which significantly differentiate early stage (stage 0) versus late stage (stage 3) breast cancer patients. In one embodiment, the method of diagnosis will employ at least one or more proteins that have increased levels, another embodiment that employs decreased levels, and yet another embodiment that employs a combination of both increased and decreased levels. In addition, the method of diagnosis may involve specific combinations of decreased expression and/or increased expression across multiple proteins in a single assay to give a pattern of protein expression changes indicative of and diagnostic for late stage (stage 3) breast cancer. The information shown along the top of Table 1 from left to right are: 1) the Uniprot accession number, 2) the log₂ ratio spectral count change between early stage (stage 0) breast cancer and late stage (stage 3) breast cancer, 3) the direction of change in late stage (stage 3) breast cancer, 4) the protein abbreviation, and 5) the name of the protein. All proteins in this list meet the criteria of P values of less than 0.05 indicating their significance, and thus identifying each of these proteins as a candidate biomarker of late stage (stage 3) breast cancer that can be used for diagnosis, prognosis, or therapeutic targets of aggressive breast cancer.

TABLE 1

| SEQ | | Log₂ | | | |
|---|---|---|---|---|---|
| ID | Uniprot | Ratio | Change in Stage | | |
| NO: | Accession | Change | 3 Breast Cancer | Abbreviation | Protein Name |
| 1 | P59998 | −1.585 | Decrease | ARPC4 | actin related protein 2/3 complex, subunit 4, 20 kDa |
| 2 | P60709 | 0.6 | Increase | ACTB | actin, beta |
| 3 | P49748 | −1.701 | Decrease | ACADVL | acyl-CoA dehydrogenase, very long chain |
| 4 | P25054 | −1.1 | Decrease | APC | adenomatous polyposis coli |
| 5 | P23526 | −1.597 | Decrease | AHCY | adenosylhomocysteinase |
| 6 | Q09666 | −1.074 | Decrease | AHNAK | AHNAK nucleoprotein |
| 7 | O43488 | −2.427 | Decrease | AKR7A2 | aldo-keto reductase family 7, member A2 (aflatoxin aldehyde |
| | | | | | reductase) |
| 8 | P25311 | −2.083 | Decrease | AZGP1 | alpha-2-glycoprotein 1, zinc-binding |
| 9 | P20073 | −0.97 | Decrease | ANXA7 | annexin A7 |
| 10 | P25705 | −0.552 | Decrease | ATP5A1 | ATP synthase, H+ transporting, mitochondrial F1 complex |
| 11 | P21810 | 2.611 | Increase | BGN | biglycan |
| 12 | P12830 | −1.569 | Decrease | CDH1 | cadherin 1, type 1, E-cadherin (epithelial) |
| 13 | Q9H251 | −1.532 | Decrease | CDH23 | cadherin-related 23 |
| 14 | P27708 | 0.959 | Increase | CAD | carbamoyl-phosphate synthetase 2 |
| 15 | P16152 | −1.462 | Decrease | CBR1 | carbonyl reductase 1 |
| 16 | Q8N3K9 | 1.3 | Increase | CMYA5 | cardiomyopathy associated 5 |
| 17 | P35221 | −1.24 | Decrease | CTNNA1 | catenin (cadherin-associated protein), alpha 1, 102 kDa |
| 18 | P35222 | −2.564 | Decrease | CTNNB1 | catenin (cadherin-associated protein), beta 1, 88 kDa |
| 19 | Q02224 | −1.822 | Decrease | CENPE | centromere protein E, 312 kDa |
| 20 | Q5SY80 | −1.585 | Decrease | C1ORF101 | chromosome 1 open reading frame 101 |
| 21 | P53621 | −1.948 | Decrease | COPA | coatomer protein complex, subunit alpha |
| 22 | P02452 | 0.942 | Increase | COL1A1 | collagen, type I, alpha 1 |
| 23 | P08123 | 1.19 | Increase | COL1A2 | collagen, type I, alpha 2 |
| 24 | P12110 | 1.091 | Increase | COL6A2 | collagen, type VI, alpha 2 |
| 25 | A6NMZ7 | 1.03 | Increase | COL6A6 | collagen, type VI, alpha 6 |
| 26 | Q99715 | 2.666 | Increase | COL12A1 | collagen, type XII, alpha 1 |
| 27 | Q07092 | 1.415 | Increase | COL16A1 | collagen, type XVI, alpha 1 |
| 28 | Q86Y22 | 1.392 | Increase | COL23A1 | collagen, type XXIII, alpha 1 |
| 29 | O00571 | −1.363 | Decrease | DDX3X | DEAD (Asp-Glu-Ala-Asp) box polypeptide 3, X-linked |
| 30 | Q96HY7 | 2.445 | Increase | DHTKD1 | dehydrogenase E1 and transketolase domain containing 1 |
| 31 | P15924 | −1.054 | Decrease | DSP | desmoplakin |
| 32 | P60981 | −1.17 | Decrease | DSTN | destrin (actin depolymerizing factor) |
| 33 | Q9UJU6 | −3.301 | Decrease | DBNL | drebrin-like |
| 34 | P24534 | −2.079 | Decrease | EEF1B2 | eukaryotic translation elongation factor 1 beta 2 |
| 35 | B3KSH1 | −1.306 | Decrease | EIF3F | eukaryotic translation initiation factor 3, subunit F |
| 36 | Q9NUQ9 | 2.713 | Increase | FAM49B | family with sequence similarity 49, member B |
| 37 | P07954 | 0.811 | Increase | FH | fumarate hydratase |
| 38 | Q9P0M6 | −0.976 | Decrease | H2AFY2 | H2A histone family, member Y2 |
| 39 | P04792 | 1.222 | Increase | HSPB1 | heat shock 27 kDa protein 1 |
| 40 | P61978 | −0.604 | Decrease | HNRNPK | heterogeneous nuclear ribonucleoprotein K |
| 41 | Q6NTA2 | −0.903 | Decrease | HNRNPL | heterogeneous nuclear ribonucleoprotein L |
| 42 | P52272 | −0.872 | Decrease | HNRNPM | heterogeneous nuclear ribonucleoprotein M |
| 43 | O43390 | −0.915 | Decrease | HNRNPR | heterogeneous nuclear ribonucleoprotein R |
| 44 | Q9BUJ2 | −1.462 | Decrease | HNRNPUL1 | heterogeneous nuclear ribonucleoprotein U-like 1 |
| 45 | P51659 | −1.948 | Decrease | HSD17B4 | hydroxysteroid (17-beta) dehydrogenase 4 |
| 46 | Q96FF7 | −2.822 | Decrease | LOC113230 | hypothetical protein LOC113230 |
| 47 | Q96HQ3 | 1.078 | Increase | MGC20647 | hypothetical protein MGC20647 |
| 48 | Q9Y4L1 | −1.429 | Decrease | HYOU1 | hypoxia up-regulated 1 |
| 49 | Q8TDY8 | −0.903 | Decrease | IGDCC4 | immunoglobulin superfamily, DCC subclass, member 4 |
| 50 | Q12905 | −1.225 | Decrease | ILF2 | interleukin enhancer binding factor 2, 45 kDa |
| 51 | Q6DN90 | 1.775 | Increase | IQSEC1 | IQ motif and Sec7 domain 1 |
| 52 | P46940 | −1.24 | Decrease | IQGAP1 | IQ motif containing GTPase activating protein 1 |
| 53 | P14923 | −1.156 | Decrease | JUP | junction plakoglobin |
| 54 | B3KY79 | 1.281 | Increase | KRT7 | keratin 7 |
| 55 | Q9Y2L5 | 1.36 | Increase | KIAA1012 | KIAA1012 |
| 56 | Q9NS87 | 1.775 | Increase | KIF15 | kinesin family member 15 |
| 57 | Q03252 | −1.552 | Decrease | LMNB2 | lamin B2 |
| 58 | A4D0S4 | 2.544 | Increase | LAMB4 | laminin, beta 4 |
| 59 | P28838 | −1.301 | Decrease | LAP3 | leucine aminopeptidase 3 |
| 60 | Q32MZ4 | −1.848 | Decrease | LRRFIP1 | leucine rich repeat (in FLII) interacting protein 1 |
| 61 | Q8N1G4 | −1.441 | Decrease | LRRC47 | leucine rich repeat containing 47 |
| 62 | P09960 | −2.128 | Decrease | LTA4H | leukotriene A4 hydrolase |
| 63 | Q14847 | −1.555 | Decrease | LASP1 | LIM and SH3 protein 1 |
| 64 | P51884 | 2.292 | Increase | LUM | lumican |
| 65 | P14174 | −2.015 | Decrease | MIF | macrophage migration inhibitory factor (glycosylation-inhibiting |
| | | | | | factor) |
| 66 | P40925 | −1.237 | Decrease | MDH1 | malate dehydrogenase 1, NAD (soluble) |
| 67 | Q9UPN3 | −1.5 | Decrease | MACF1 | microtubule-actin crosslinking factor 1 |
| 68 | P98088 | 1.316 | Increase | MUC5AC | mucin 5AC, oligomeric mucus/gel-forming |
| 69 | P55198 | 1.29 | Increase | MLLT6 | myeloid/lymphoid or mixed-lineage leukemia (homolog, |
| | | | | | Drosophila) |
| 70 | O00567 | −1.684 | Decrease | NOP56 | NOP56 ribonucleoprotein homolog (yeast) |
| 71 | Q14980 | −0.899 | Decrease | NUMA1 | nuclear mitotic apparatus protein 1 |
| 72 | Q96L73 | −1.063 | Decrease | NSD1 | nuclear receptor binding SET domain protein 1 |
| 73 | P19338 | −0.311 | Decrease | NCL | nucleolin |
| 74 | Q99497 | −1.435 | Decrease | PARK7 | Parkinson disease (autosomal recessive, early onset) 7 |
| 75 | P23284 | −1.252 | Decrease | PPIB | peptidylprolyl isomerase B (cyclophilin B) |
| 76 | Q15063 | 1.745 | Increase | POSTN | periostin, osteoblast specific factor |
| 77 | Q5VU43 | −1.948 | Decrease | PDE4DIP | phosphodiesterase 4D interacting protein |
| 78 | Q99623 | −0.934 | Decrease | PHB2 | prohibitin 2 |
| 79 | Q9UQ80 | −2.865 | Decrease | PA2G4 | proliferation-associated 2G4, 38 kDa |
| 80 | Q14914 | −1.585 | Decrease | PTGR1 | prostaglandin reductase 1 |
| 81 | Q16401 | −1.5 | Decrease | PSMD5 | proteasome (prosome, macropain) 26S subunit, non-ATPase, 5 |
| 82 | P49720 | −1.289 | Decrease | PSMB3 | proteasome (prosome, macropain) subunit, beta type, 3 |
| 83 | P30101 | −0.745 | Decrease | PDIA3 | protein disulfide isomerase family A, member 3 |
| 84 | P14618 | −0.577 | Decrease | PKM2 | pyruvate kinase, muscle |
| 85 | P52566 | −1.1 | Decrease | ARHGDIB | Rho GDP dissociation inhibitor (GDI) beta |
| 86 | P49247 | 2.576 | Increase | RPIA | ribose 5-phosphate isomerase A |
| 87 | P36578 | −1.822 | Decrease | RPL4 | ribosomal protein L4 |
| 88 | P62249 | −2.948 | Decrease | RPS16 | ribosomal protein S16 |
| 89 | P39019 | −2.211 | Decrease | RPS19 | ribosomal protein S19 |
| 90 | P15880 | −1.314 | Decrease | RPS2 | ribosomal protein S2 |
| 91 | Q9NQ03 | −1.128 | Decrease | SCRT2 | scratch homolog 2, zinc finger protein (Drosophila) |
| 92 | O75396 | −0.933 | Decrease | SEC22B | SEC22 vesicle trafficking protein homolog B (S. cerevisiae) |
| 93 | A6PVW9 | −1.421 | Decrease | SELENBP1 | selenium binding protein 1 |
| 94 | Q15019 | −2.363 | Decrease | SEPT2 | septin 2 |
| 95 | O75368 | −2.363 | Decrease | SH3BGRL | SH3 domain binding glutamic acid-rich protein like |
| 96 | Q8WXH0 | −0.893 | Decrease | SYNE2 | spectrin repeat containing, nuclear envelope 2 |
| 97 | Q01082 | −1.069 | Decrease | SPTBN1 | spectrin, beta, non-erythrocytic 1 |
| 98 | Q15393 | −1.244 | Decrease | SF3B3 | splicing factor 3b, subunit 3, 130 kDa |
| 99 | Q9UMS6 | −2.948 | Decrease | SYNPO2 | synaptopodin 2 |
| 100 | P10599 | 0.891 | Increase | TXN | thioredoxin |
| 101 | P07996 | 1.985 | Increase | THBS1 | thrombospondin 1 |
| 102 | P19971 | −1.421 | Decrease | TYMP | thymidine phosphorylase |
| 103 | Q8WZ42 | −0.372 | Decrease | TTN | titin |
| 104 | B3KPZ8 | −0.82 | Decrease | TKT | transketolase |
| 105 | Q9UHN6 | 1 | Increase | TMEM2 | transmembrane protein 2 |
| 106 | A6NKL6 | 0.637 | Increase | TMEM200C | transmembrane protein 200C |
| 107 | P31946 | −0.773 | Decrease | YWHAB | tyrosine 3-monooxygenase/tryptophan 5-monooxygenase |
| | | | | | activation protein |
| 108 | Q70EL4 | 1.173 | Increase | USP43 | ubiquitin specific peptidase 43 |
| 109 | P22314 | −0.744 | Decrease | UBA1 | ubiquitin-like modifier activating enzyme 1 |
| 110 | P56704 | 0.814 | Increase | WNT3A | wingless-type MMTV integration site family, member 3A |
| 111 | P13010 | −1.158 | Decrease | XRCC5 | X-ray repair complementing defective repair in Chinese hamster |
| | | | | | cells 5 |
| 112 | P12956 | −0.887 | Decrease | XRCC6 | X-ray repair complementing defective repair in Chinese hamster |
| | | | | | cells 6 |
| 113 | Q5TAX3 | −0.217 | Decrease | ZCCHC11 | zinc finger, CCHC domain containing 11 |

The present methods encompass not only methods of diagnosis, prognosis, therapeutic treatment, and compositions that employ the proteins recited in Table 1, but also those that employ related proteins. In one embodiment, the related proteins encompass proteins/polypeptides that share at least some amino acid sequence with the proteins in Table 1, and which are produced by translation of alternate transcripts (or alternately processed transcripts) from the genes encoding the proteins in Table 1. In another embodiment, related proteins encompasses proteins/polypeptides that share at least some amino acid sequence with the proteins in Table 1 produced by changes at the translational or post-translation level (e.g., post translational modifications). In either embodiment, related proteins may comprise a sequence of greater than five, six, seven, eight, ten, twelve, fifteen, eighteen, or twenty contiguous amino acids that is identical to a sequence found in a protein in Table 1.

Embodiments provided herein include compositions comprising one or more, two or more, three or more, four or more, five or more, six or more, eight or more, or ten or more of the proteins in Table 1, or polypeptide fragments thereof. In some embodiments, the compositions comprise two or more, three or more, four or more, five or more, six or more, or seven or more antibodies that bind specifically to proteins found in Table for peptide fragments of those proteins. Compositions comprising peptides may include one or more, two or more, three or more, four or more, five or more, six or more, eight or more, or ten or more peptides that are isotopically labeled. Each of the peptides may be labeled with one or more isotopes selected independently from the group consisting of: ¹⁸O, ¹⁷O, ³⁴S, ¹⁵N, ¹³C, ²H or combinations thereof. Compositions comprising peptides from the any of the proteins in Table 1, whether isotope labeled or not, do not need to contain all of the peptides from any given protein (e.g., a complete set of tryptic peptides). In some embodiments the compositions will comprise only one, two, three, four, five, six, or seven peptides for two, three, four, five, six, seven, eight, nine, ten, or more of the proteins appearing in Table 1 or Table 2. Compositions comprising peptides may be in the form of dried or lyophilized materials, liquid (e.g., aqueous) solutions or suspensions, arrays, or blots.

### Use of the Biomarkers

The protein biomarkers described herein can be advantageous employed to improve the treatment of patients with breast cancer. The over-expression and/or under-expression of one or more proteins in late stage (stage 3) breast cancer vs. early stage (stage 0) breast cancer and the ability to assay for this over-expression and/or under-expression in a biological sample can be used to determine whether or not a person with breast cancer has a type of cancer that is more aggressive than others and should be treated as such. Where a protein profile is prepared that suggests a patient has a form of breast cancer that is more aggressive, the results may also indicate choices for therapy and/or treatment regimens which are different from those that would be used for patients with less aggressive breast cancers. In addition, determinations based on the altered expression of multiple proteins are more likely to be effective as indicators of late stage (stage 3) breast cancer than assessment of one or two proteins individually. The present methods include and provide for assessment and correlation of multiple proteins simultaneously in a single biological sample from an individual suspected of being afflicted with an aggressive form of breast cancer.

Over-expression and/or under-expression of one or more proteins in late stage (stage 3) breast cancer vs. early stage (stage 0) breast cancer and the ability to assay for this over-expression and/or under-expression in a biological sample can be used to help determine which therapeutic agent is chosen to achieve the best course of disease treatment. One or more of the proteins indicated herein can be targeted directly with a drug so that breast cancer cells can be killed preferentially instead of the normal cells in the tissue that are not expressing one or more of these proteins.

The type of biological sample assayed using one or more of these proteins as biomarkers of recurrent breast cancer include biopsied tissue or tissue removed surgically. The tissue can be fresh, frozen, and/or chemically fixed such as that which is preserved in formalin and other chemical fixatives of the like. Another form the biological sample can take is fractionated or unfractionated biofluid samples such as serum, plasma, whole blood, lymph fluids, and ascites fluids. All of these forms of a patient-derived biological sample can be assayed for expression of one or more of the proteins in Table 1.

Because both nucleic acids and protein can be analyzed from the same biomolecular lysate preparations employed herein (e.g. as U.S. Pat. No. 7,473,532) it is possible to generate additional information about disease diagnosis and drug treatment decisions from the same sample. For example, additional information about the state of the cells and their potential for uncontrolled growth, potential drug resistance, and the development of cancers can be obtained by analyzing nucleic acids from those lysate preparations. By using the lysate preparations for both protein/peptide analysis and nucleic acid analysis it is possible to obtain information about the status of any one, two, three, four, five or more genes and/or the nucleic acids, and/or the proteins they encode (e.g., mRNA molecules and their expression levels or splice variations) from the same biomolecular lysate preparation. For example information about any one, two, three, four, five or more peptides in Table 1, and or the proteins from which they were derived or the nucleic acids encoding those proteins may be assessed. The nucleic acids can be examined, for example, by: one or more sequencing methods, conducting restriction fragment polymorphism analysis, conducting hybridization with another nucleic acid, identify deletion, insertions, and/or determining the presence of mutations, including but not limited to, single base pair polymorphisms, transitions and/or transversions. Such tests may be conducted in any suitable format including, but not limited to, arrays, microarrays, on blots, or in solution (e.g., by polymerase chain reaction “PCR” or ligase chain reaction “LCR”).

Where hybridization with another nucleic acid is employed, the assay or test may be conducted in any suitable format (e.g., arrays/microarrays, blots, and the like) by contacting nucleic acids under conditions of suitable stringency to obtain specific binding. The required “stringency” of hybridization reactions determinable by one of ordinary skill in the art, and generally involves an empirical calculation dependent upon probe length, washing temperature, and salt concentration. In general, longer probes require higher temperatures for proper annealing, while shorter probes need lower temperatures. Hybridization generally depends on the ability of denatured DNA to anneal when complementary strands are present in an environment below their melting temperature, with. The higher the degree of desired homology between the probe and hybridizable sequence, the higher the relative temperature which can be used, and higher relative temperatures tend to make hybridization reactions more stringent and vice versa. See e.g., Ausubel et al., Current Protocols in Molecular Biology, Wiley Interscience Publishers, (1995). Hybridization reactions will typically employ stringent conditions or moderately stringent conditions.

“Stringent conditions” typically employ low ionic strength with or without a denaturant (e.g., formamide) and high temperature for washing, for example, 0.015 M sodium chloride/0.0015 M sodium citrate/0.1% sodium dodecyl sulfate at 50° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing in 1×SSC at about 37-50° C. The skilled artisan will recognize how to adjust the temperature, ionic strength, etc. as necessary to accommodate factors such as probe length and the like.

In one embodiment, samples are analyzed for one, two, three, four, five, six, seven, eight, nine or more peptides produced from the proteins in Table 1, and/or nucleic acids encoding one or more of those peptides or the proteins from which they were derived by proteolysis. In an embodiment, samples are analyzed for two, three, four, five, six, seven, eight, nine or more peptides produced from the proteins in Table 1, and/or two, three, four, five, six, seven, eight, nine or more nucleic acids encoding proteins from Table 1, where the proteins from Table 1 are selected from any range of proteins represented by SEQ ID Nos: 1-50, 51-113, 1-25, 26-50, 51-75, 76-100, 1-10, 11-20, 21-30, 31-40, 41-50, 51-60, 61-70, 71-80, 81-90, 91-100, or 101-113 of Table 1.

### Example

Seven (7) formalin fixed breast cancer tissue samples obtained from patients whose breast cancer was histologicaly categorized as early stage (stage 0) primary breast cancer and nine (9) breast cancer tissue samples from patients whose breast cancers were histologicaly categorized as late stage (stage 3) primary breast cancer were interrogated for differential protein expression that correlates to cancer, and where these proteins may be used to improve diagnosis, prognosis, and therapy of breast cancer.

Tissue sections were prepared from each tissue for histologic analysis and procurement of epithelial cancer cells was performed by tissue microdissection. Soluble protein lysates were prepared from microdissected breast cancer tissue samples using the Liquid Tissue™ MS Protein Prep Kit (Expression Pathology, Inc.). Each lysate consisted of the total protein content of the microdissected cells digested into predictable peptide fragments by the protease trypsin. In this form each and every protein lysate can be evaluated by the technology of mass spectrometry for identification and quantification of the proteins present in each lysate. In addition, the total mass spectrometry data across all samples is used to determine differential protein expression between individual samples and between primary tumors from early stage (stage 0) breast cancer patients and primary tumors from patients with late stage (stage 3) breast cancer.

Mass spectrometry analysis of each trypsin-digested protein lysate was performed according to the following. Liquid chromatography (LC) was performed using a Dionex Ultimate 3000 system coupled on-line to a ThermoFisher linear ion trap mass spectrometer (MS). Separation of the sample was performed using a 75 μm ID×360 μm OD×10-cm-long fused silica capillary column 5 μm, 300 Å pore size Jupiter C-18 stationary phase. After injecting 5 μl of re-suspended protein lysate, the column was washed with 98% mobile phase A (0.1% formic acid in water) for 30 min and peptides were eluted using a linear gradient of 2% mobile phase B (0.1% formic acid in acetonitrile) to 42% mobile phase B in 140 min, then to 98% B in an additional 20 min, all at a constant flow rate of 250 nL/min. The Linear Ion Trap Mass Spectrometer (LIT-MS) was operated in a data-dependent MS/MS mode in which each full MS scan (precursor ion selection scan range of m/z 350-1800) was followed by seven MS/MS scans where the seven most abundant peptide molecular ions were selected for tandem MS using a relative collision-induced dissociation (CID) energy of 35%. Dynamic exclusion was utilized to minimize redundant selection of peptides for CID.

Peptide identifications were obtained by searching the LC-MS/MS data utilizing SEQUEST (BioWorks, v3.2, ThermoScientific) on a 72-node Beowulf cluster against a UniProt-derived human proteome database (version 10/08, 56,301 protein entries) obtained from the European Bioinformatics Institute (EBI) using the following parameters: trypsin (KR); full enzymatic-cleavage; two missed cleavages sites; 1.5 Da peptide mass tolerance peptide tolerance, 0.5 Da fragment ion tolerance and variable modifications for methionine oxidation (m/z 15.99492). Resulting peptide identifications were filtered according to specific SEQUEST scoring criteria: delta correlation (ΔCₙ) ≧0.08 and charge state dependent cross correlation (Xcorr) ≧1.9 for [M+H]¹⁺, ≧2.2 for [M+2H]²⁺, and ≧3.5 for [M+3H]³⁺ (Supplemental Table 1). These criteria resulted in a false discovery rate (FDR) of 5.84% for all peptides identified as determined by searching the entire data set against a decoy human database where the protein sequences were reversed. Protein abundance was derived by spectral counting (SC) and peptides whose sequences mapped to multiple protein isoforms were grouped as per the principle of parsimony. To determine statistically significant changes in protein abundance across patient samples by disease stage sub-groups, a hierarchical supervised cluster analysis of peptides identified from early stage (stage 0) versus late stage (stage 3) cancer was performed in which the variance in total spectral count peptides identified was determined utilizing the Mann-Whitney rank-sum test (significance level p≦0.05, Fisher's exact test) paired with the filter criteria requiring that 60% of the samples in a supervised group had a minimum peptide count of 2 or greater for a given protein.

Using the high confidence peptide data, peptide lists for each sample were combined and redundant peptide identifications were eliminated to generate a list of unique peptides. Each peptide in the list was already associated with a protein, so that the list was easily converted to a list of proteins, specifically a list of unique proteins was created for each patient sample. Based on these data a quantitative analysis to determine differential protein expression between cancer and normal was performed using the Spectral Count Quantitation method. Spectral Count Quantitation is the process of counting the number of unique peptides associated with each protein. A value of 4 beside a protein name reflects that there were 4 unique peptides that were associated with that particular protein. There may have been numerous repeated identifications for any of the individual peptides but, the count was based on unique peptides and not total peptides. This count directly correlates to the relative abundance of each particular protein, thus the more unique peptides identified for a proteins the greater the relative expression of that protein in any particular sample.

It was the goal of this data analysis to identify those proteins whose derived quantitative expression levels showed significant differences in expression between early stage (stage 0) breast cancer and late stage (stage 3) breast cancer samples. These criteria were established because those proteins that are identified by greater numbers of unique peptides in early stage (stage 0) breast cancer cells over late stage (stage 3) breast cancer cells are the most likely candidates for new biomarkers of aggressive, dangerous breast cancer. Cluster analysis, which is a statistical method that determines which items are significantly different between 2 separate groups, identified 113 proteins differentially expressed between early stage (stage 0) breast cancer cells and late stage (stage 3) breast cancer, and which are listed in Table 1.

### Selected Reaction Monitoring Assay for Analysis of Patient Tissue

The SRM/MRM assays described herein can measure relative or absolute quantitative levels of one or more specific peptides derived from one or more of the proteins listed in Table 1. The method is utilized to provide a means of measuring the amount of a given peptide, peptides, protein, or proteins, by mass spectrometry in a given peptide/protein preparation obtained from a patient's biological sample such as bodily fluid or a Liquid Tissue™ lysate from formalin fixed paraffin embedded tissue. SRM/MRM assays can measure peptides directly in complex protein lysates prepared from cells procured from patient tissue samples, such as formalin fixed cancer patient tissue.

Methods of preparing protein samples from formalin-fixed tissue are described in U.S. Pat. No. 7,473,532, the contents of which are hereby incorporated by references in their entirety. The methods described in that patent may conveniently be carried out using Liquid Tissue™ reagents available from Expression Pathology, Inc. (Rockville, Md.).

Results from SRM/MRM assays can be used to correlate accurate and precise quantitative levels of a given peptide, peptides, protein, or proteins, with the specific cancer of the patient from whom the biological sample was collected. This not only provides diagnostic information about the cancer, but also permits a physician or other medical professional to determine appropriate therapy for the patient. Such an assay provides diagnostically and therapeutically important information about levels of protein expression in a diseased tissue or other patient sample such as bodily fluids is termed a companion diagnostic assay. For example, such an assay can be designed to diagnose the stage or degree of a cancer and determine which therapeutic agent, or course of therapy, to which a patient is most likely to respond with a positive outcome. An SRM/MRM assay measures relative or absolute levels of specific unmodified peptides from a given protein, or protein, and also can measure absolute or relative levels of specific modified peptides from proteins. Examples of modifications include phosphorylated amino acid residues and glycosylated amino acid residues that are present on the peptides.

Relative quantitative levels of a given peptide, peptides, protein, or proteins, are determined by the SRM/MRM methodology whereby the mass spectrometry-derived signature peak area (or the peak height if the peaks are sufficiently resolved) of an individual peptide, or multiple peptides, from a given protein, or proteins, in one biological sample is compared to the signature peak area determined for the same identical peptide, or peptides, from the same protein, or proteins, using the same methodology in one or more additional and different biological samples. In this way, the amount of a particular peptide, or peptides, from a given protein, or proteins, is determined relative to the same peptide, or peptides, from the same protein, or proteins, across 2 or more biological samples under the same experimental conditions. In addition, relative quantitation can be determined for a given peptide, or peptides, from a single protein within a single sample by comparing the signature peak area for that peptide for that given protein by SRM/MRM methodology to the signature peak area for another and different peptide, or peptides, from a different protein, or proteins, within the same protein preparation from the biological sample. In this way, the amount of a particular peptide from a given protein, and therefore the amount of the given protein, is determined relative one to another within the same sample. These approaches generate quantitation of an individual peptide, or peptides, from a given protein to the amount of another peptide, or peptides, between samples and within samples wherein the amounts as determined by signature peak area are relative one to another, regardless of the absolute weight to volume or weight to weight amounts of peptides in the protein preparation from the biological sample. Relative quantitative data about individual signature peak areas between different samples are normalized to the amount of protein analyzed per sample. Relative quantitation can be performed across many peptides simultaneously in a single sample and/or across many samples to gain insight into relative protein amounts, one peptide/protein with respect to other peptides/proteins.

Absolute quantitative levels of a given protein, or proteins, are determined by the SRM/MRM methodology whereby the SRM/MRM signature peak area of an individual peptide from a given protein in one biological sample is compared to the SRM/MRM signature peak area of a known amount of a “spiked” internal standard. In one embodiment, the internal standard is a synthetic version of the same exact peptide that contains one or more amino acid residues labeled with one or more heavy isotopes. Such isotope labeled internal standards are synthesized so that mass spectrometry analysis generates a predictable and consistent SRM/MRM signature peak that is different and distinct from the native peptide signature peak, and which can be used as a comparator peak. Thus when the internal standard is spiked in known amounts into a protein preparation from a biological sample and analyzed by mass spectrometry, the signature peak area of the native peptide is compared to the signature peak area of the internal standard peptide, and this numerical comparison indicates either the absolute molarity and/or absolute weight of the native peptide present in the original protein preparation from the biological sample. Absolute quantitative data for fragment peptides are displayed according to the amount of protein analyzed per sample. Absolute quantitation can be performed across many peptides, and thus proteins, simultaneously in a single sample and/or across many samples to gain insight into absolute protein amounts in individual biological samples and in entire cohorts of individual samples.

The SRM/MRM assay method can be used to aid diagnosis of the stage of cancer, for example, directly in patient-derived tissue, such as formalin fixed tissue, and to aid in determining which therapeutic agent, and/or treatment strategy, would be most advantageous for use in treating that patient. Cancer tissue that is removed from a patient either through surgery, such as for therapeutic removal of partial or entire tumors, or through biopsy procedures conducted to determine the presence or absence of suspected disease, is analyzed to determine whether or not a specific protein, or proteins, and which forms of proteins, are present in that patient tissue. Moreover, the expression level of the protein(s) can be determined and compared to a “normal” or reference level found in healthy tissue or tissue that shows a different stage/grade of cancer. This information can then be used to assign a stage or grade to a specific cancer and can be matched to a strategy for treating the patient based on the determined levels of specific proteins. Matching specific information about levels of a given protein, or proteins, as determined by an SRM assay, to a treatment strategy that is based on levels of these proteins in cancer cells derived from the patient defines what has been termed a personalized medicine approach to treating disease. The SRM/MRM assay method described herein form the foundation of a personalized medicine approach by using analysis of proteins from the patient's own tissue as a source for diagnostic and treatment decisions. The SRM/MRM method described herein can be used to specifically assay proteins in Table 1.

Although the invention has been described in relation to certain embodiments thereof, and many details have been set forth for purposes of illustration, it will be apparent to those skilled in the art that the inventions is susceptible to additional embodiments and that certain of the details described herein may be varied considerably without departing from the basic principles of the inventions described herein.

1. A method of diagnosing that a breast cancer as an early stage primary breast cancer (stage 0) or a late stage (stage 3) breast cancer comprising the steps of:
a) measuring the level of expression of at least one or more, at least two or more, at least 3 or more, or multiples and combinations of the proteins listed in Table 1 in a sample from a human patient, in which said sample comprises breast cancer tissue, breast cancer cells, or a bodily fluid such as blood or ascites fluid containing proteins from said patient's breast cancer said sample; and
b) determining increased expression and/or decreased expression of said at least one or more, at least two or more, at least 3 or more, or multiples and combinations of the proteins listed in Table 1 in a late stage (stage 3) breast cancer as compared to expression levels of said at least one or more, at least two or more, at least 3 or more, or multiples and combinations of the proteins listed in Table 1 in early stage (stage 0) breast cancer indicating the potential that a primary breast cancer is more or less aggressive in said patient.

2. The method of claim 1, wherein said breast cancer sample consists essentially of breast epithelial cells.

3. The method of claim 1, wherein said bodily fluids include but are not limited to fractionated or unfractionated blood, serum, plasma, lymphatic fluid, or fluid collected by pleural effusion.

4. The method of claim 1, wherein the tissue is collected by biopsy or surgical procedure.

5. The method of claim 4, wherein the tissue is chemically fixed and preserved.

6. The method of claim 5, wherein said chemical fixation and preservation comprises formalin fixation and embedding in paraffin.

7. The method of claim 4, wherein the tissue is frozen.

8. The method of claim 1, wherein said proteins are measured as intact, full-length proteins or are measured by measuring multiple or individual peptides derived by fragmentation of the intact, full-length proteins.

9. The method of claim 1, wherein said proteins are detected by mass spectroscopy and the level of measured expression of said proteins is determined by spectral count quantification after said mass spectroscopy

10. The method of claim 1, wherein said proteins are detected by mass spectroscopy and the level of measured expression of said proteins is determined by a Selected Reaction Monitoring (SRM) assay.

11. The method claim 1, wherein said proteins are detected by mass spectroscopy and the level of measured expression of said proteins is determined by a multiplex SRM assay, termed a multiple reaction monitoring (MRM) assay where more than one protein is detected and quantitated in a single mass spectrometry analysis.

12. The method of claim 8, wherein said mass spectroscopy is selected from the group consisting of LC-ESI-MS/MS, MALDI-MS, tandem MS, TOF/TOF, TOF-MS, TOF-MS/MS, triple quadrupole MS, and triple quadrupole MS/MS.

13. The method of claim 12, wherein said mass spectroscopy comprises liquid chromatography-tandem mass spectroscopy.

14. The method of claim 1, wherein said proteins are detected and their levels of expression are determined by a protein microarray or by an immunoassay.

15. The method of claim 14, wherein said immunoassay is selected from the group consisting of immunohistochemistry, Western blot, dot blot, and ELISA.

16. A method of indicating choice of therapy of primary breast cancer, comprising the steps of:
a) detecting the presence and measuring the level of expression of at least one or more, at least two or more, at least 3 or more, or multiples and combinations of the proteins listed in Table 1 in a sample from a human patient, in which said sample comprises breast cancer tissue, breast cancer cells, or a bodily fluid such as blood or ascites fluid containing proteins from said patient's breast cancer said sample; and
b) determining increased expression and/or decreased expression of said at least one or more, at least two or more, at least 3 or more, or multiples and combinations of the proteins listed in Table 1 in a late stage (stage 3) breast cancer as compared to expression levels of said at least one or more, at least two or more, at least 3 or more, or multiples and combinations of the proteins listed in Table 1 in early stage (stage 0) breast cancer indicating the potential that a primary breast cancer is more or less aggressive in said patient.

17. A method comprising quantifying the amount of one or more, two or more, three or more, four or more, five or more, six or more, seven or more, or eight or more of the proteins in Table 1 or peptide fragments thereof.

18. A composition comprising one or more, two or more, three or more, four or more, five or more, six or more, seven or more, eight or more, or ten or more of the proteins in Table 1, peptides thereof, or antibodies thereto.

19. The composition of claim 18, comprising one or more, two or more, three or more, four or more, five or more, six or more, seven or more, or eight or more peptides of proteins in Table 1, wherein each peptide is derived from a different protein.

20. The composition of claim 19, wherein each of the peptides is labeled with one or more isotopes independently selected from the group consisting of: 18O, 17O, 34S, 15N, 13C, 2H or combinations thereof.

21. The composition of claim 19, comprising one or more, two or more, three or more, four or more, five or more, six or more, seven or more, or eight or more peptides of proteins in Table 1 that are increased in tissues from primary tumors that are late stage (stage 3) breast cancers.

22. The composition of claim 19, comprising one or more, two or more, three or more, four or more, five or more, six or more, seven or more, or eight or more peptides of proteins in Table 1 that are decreased in tissues from primary tumors that are late stage (stage 3) breast cancers.

23. The composition of claim 21, comprising one or more, two or more, three or more, four or more, five or more, six or more, seven or more, or eight or more peptides of proteins in Table 1 that are decreased in tissues from primary tumors that recurred in two years

24. The method of claim 1, further comprising assessing and/or determining the level (amount) or sequence of one, two, three, four, five, six, seven, eight nine, ten or more nucleic acids in said protein digest.

25. The method of claim 24, wherein said nucleic acids have a length selected independently from greater than about: 15, 20, 25, 30, 35, 40, 50, 60, 75, or 100 nucleotides in length.

26. The method of claim 25, wherein said nucleic acids have a length selected independently from less than about: 150, 200, 250, 300, 350, 400, 500, 600, 750, 1,000, 2,000, 4,000, 5,000, 7,500, 10,000, 15,000, or 20,000 nucleotides in length.

27. The method of claim 24, wherein assessing and/or determining the level (amount) or sequence comprises, determining either the sequence of nucleotides in the nucleic acids and/or a characteristic of the nucleic acids by any one or more of: nucleic acid sequencing, conducting restriction fragment polymorphism analysis, conducting hybridization with another nucleic acid, identification of one or more deletions and/or insertions, and/or determining the presence of mutations, including but not limited to, single base pair polymorphisms, transitions and/or transversions.

28. The method of claim 24, wherein one, two, three, four, five, six, seven, eight nine, ten or more nucleic acids encode for proteins in Table 1.

29. The method of claim 26, wherein said nucleic acids encode for proteins of SEQ ID Nos: 1-50, 51-113, 1-25, 26-50, 51-75, 76-100, 1-10, 11-20, 21-30, 31-40, 41-50, 51-60, 61-70, 71-80, 81-90, 91-100, 101-113 or fragments thereof Table 1.
